(19) United States
(12) Patent Application Publication
(10) Pub. No.: US 2012/0150319 A1
(43) Pub. Date: Jun. 14, 2012

(54) LIMB PROSTHESIS WITH A SEALING MEMBER BETWEEN A LINER AND A SOCKET

(76) Inventors: Laszlo DALLOS, (US); Lynn SNYDER, (US)

(21) Appl. No.: 13/325,556

(22) Filed: Dec. 14, 2011

Related U.S. Application Data
(60) Provisional application No. 61/422,815, filed on Dec. 14, 2010.

(51) Int. Cl. A61F 2/78

(52) U.S. Cl. 623/36

(57) ABSTRACT

A limb prosthesis includes a liner, a shell, and at least one sealing member. The at least one sealing member is secured to and circumferentially surrounds the liner. The at least one sealing member is disposed between the liner and the shell and enhances security of the attachment of the shell to the liner.

### CROSS-REFERENCE TO RELATED APPLICATIONS

This application claims priority to U.S. Provisional Patent Application No. 61/422,815 filed 14 Dec. 2010, which application is herein expressly incorporated by reference.

### FIELD

The present teachings generally relate to prostheses for limbs such as arms and legs. The present teachings more particularly relate to a limb prosthesis having a liner and a socket and a sealing member positioned between the liner and the socket for enhancing security of the prosthetic socket. The present teachings also more particularly relate to a method of providing an improved liner/socket interface and thereby improving suction and/or mechanical suspension and reducing pistoning and rotation of the prosthesis.

### BACKGROUND

Various prosthetic devices for limbs such as legs and arms are known in the art. Known limb prostheses may generally include a liner and a shell socket. The liner is attached to the severed limb and the shell socket may be secured to the liner with suction. The remainder of the prosthesis may be attached to the shell socket.

While significant advancements have been made in the field of limb prosthetics in recent years, a need remains in the art for improved techniques for securing prostheses to the body.

### SUMMARY

The present teachings provide a limb prosthesis having improved security. The present teachings also provide a limp prosthesis securable to a limb with reduced pistoning and rotation relative to the limb.

According to one particular aspect, the present teachings provide a limb prosthesis including a liner, a shell, and at least one sealing member. The at least one sealing member may be secured to and may circumferentially surround the liner. The at least one sealing member is disposed between the liner and the shell and may enhance security of the attachment of the shell to the liner.

According to another particular aspect, the present teachings provide a limb prosthesis including a liner and a hollow shell. The hollow shell has a proximal end and a distal end. The proximal end is open to receive the liner. The limb prosthesis may further include at least one band-shaped sealing member attached to and circumferentially surrounding the liner. The at least one sealing member may be disposed between the liner and the shell.

According to yet another particular aspect, the present teachings provide a method of securing a limb prosthesis to a patient. The method includes securing a liner to the patient. The method may additionally include placing at least one band circumferentially around the liner. The method may further include attaching a hollow shell to the liner by placing the hollow shell at least partially over the liner such that the at least one band is between the liner and the hollow shell.

According to still yet another aspect, the present teachings provide a kit for securing a hollow shell of a limb prosthesis to a liner of the prosthesis. The kit may include at least one sealing member for circumferentially surrounding the liner. The at least one sealing member may have a band-shape and may be adapted to be disposed between the liner and the shell. An adhesive may secure the at least one sealing member to the liner. The at least one sealing member may enhance security of the attachment of the shell to the liner.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DRAWINGS

The present invention will become more fully understood from the detailed description and the accompanying drawings, wherein:

FIG. 1 is perspective, environmental view illustrating a limb prosthesis according to the present teachings, the limb prosthesis shown operatively associated with a patient.

FIG. 2 is a side view of the limb prosthesis of FIG. 1, illustrating the shell socket removed therefrom for purposes of illustration, the shell socket shown operatively associate with a sealing member.

FIG. 3 is a side view similar to FIG. 2, illustrating the prosthetic liner operatively associated with a plurality of sealing members.

FIG. 4 is an enlarged side view of the sealing member of FIG. 2 shown removed from the liner for purposes of illustration.

### DETAILED DESCRIPTION

The following description of various aspects of the present invention is merely exemplary in nature and is in no way intended to limit the invention, its application, or uses.

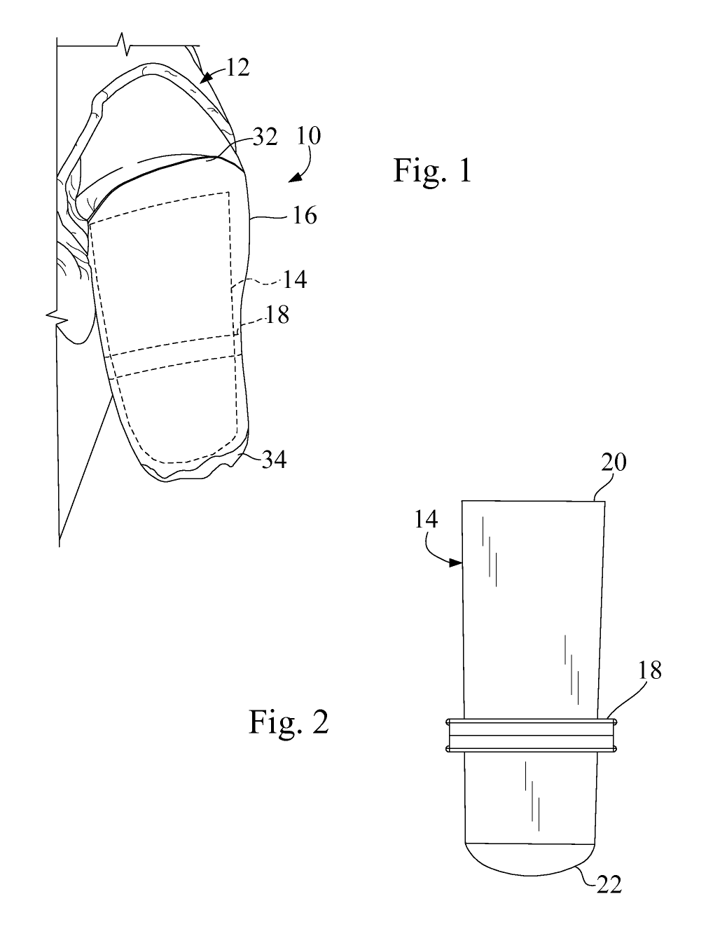

With general reference to the drawings, a sub-assembly of a limb prosthesis constructed in accordance with the present teaching is illustrated and generally identified at reference character 10. In FIG. 1, the sub-assembly 10 is shown operatively associated with a limb of a patient 12. As particularly shown in the drawings, the present teachings may be used with an upper arm prosthesis. It will be appreciated, however, that the present teachings are not so limited. In this regard, the present teachings have application for various other types of prostheses. For example, the present teachings may alternatively be used in connection with above the knee prostheses, below the knee prostheses, and other prostheses.

The sub-assembly 10 may generally include a liner 14 and a shell or socket shell 16. FIG. 1 illustrates the liner 14 and the shell 16 operatively associated with the patient 12. FIG. 2 illustrates the shell 16 removed from the liner 14. It will be understood by those skilled in the art that attachment of the remainder of the prosthesis (not shown) is conventional insofar as the present disclosure is concerned.

As shown in FIG. 1, the sub-assembly of the limb prosthesis or prosthesis 10 may include one or more sealing members 18 positioned between the liner 14 and the socket shell 16. It will be understood that the liner 14 and socket shell 16 are conventional insofar as the present teachings are concerned. In this regard, the present teachings may be utilized with various other prostheses having a liner 14 and a socket shell 16. As will be understood further below, the present teachings are more particularly directed to a sealing member 18 positioned between the liner 14 and the socket shell 16 and a related method.

Before addressing the sealing member 18 and related aspects, a brief understanding of the environment illustrated in the drawings is warranted. The liner 14 may be unitarily formed to define a main body portion having a proximal end 20 and a distal end 22. The main body portion is hollow and the proximal end 20 is open for attachment to the limb of the patient 12. The distal end 22 is closed.

The shell 16 may be a hollow shell having a proximal end 32 and a distal end 34. The proximal end 32 may be open to receive the liner 14. While not illustrated, the shell 16 may conventionally include a valve (not shown) proximate to the distal end 34 for releasing air from an interior of the hollow shell 16. The valve may be a conventional one-way valve that functions to permit air to vent from between the shell 16 and the liner 14.

As illustrated in FIG. 2, the prosthesis sub-assembly 10 may include a single sealing member 18. The sealing member 18 may be ring or band shaped. The sealing member 18 may be secured to and circumferentially surround the liner 14. The sealing member 18 may be disposed between the liner 14 and the shell 16 along a length of the liner 14.

In one particular application, the sealing member 18 may be constructed of silicone. In the exemplary embodiment, the sealing member 18 is both flexible and stretchable. Other materials may also be used within the scope of the present teachings.

The sealing member 18 is sized and shaped to provide a sufficient seal between the liner 14 and the shell 16. In a relaxed state, an inner diameter of the sealing member 18 may be slightly undersized in circumference compared to an outer diameter of the liner 14. The sealing member 18 may have a width W greater than a thickness. In one particular application, the sealing member 18 has an inner diameter with a relaxed circumference C ranging from approximately 14 cm to approximately 40 cm depending on the circumference of the liner 14. It will be understood that the relaxed circumference may be slightly smaller than the outer circumference of the liner 14 at the point of the liner 14 intended to receive the sealing member 18.

As perhaps best illustrated in FIG. 4, the sealing member 18 may be formed to include a main body portion, a proximal portion 40 and a distal portion 42. The proximal and distal portions 40 and 42 may radially extend outward beyond the main body portions and thereby define circumferentially extending ribs. As illustrated, the ribs 40 and 42 extend completely about the sealing member 18 in a circumferentially direction. In other applications, the ribs 40 and 42 may only extend partially around the sealing member 18. Areas of increased thickness may define ribs (outer ribs) are designed to compress against inner surface of socket to enhance sealing effect of the ring.

The sealing member 18 may have a width of about 3.1 cm between outermost edges and 2.1 cm in area between outer ribs (areas of increased thickness) and a thickness ranging from about 2 mm to about 4 mm. It will be understood that the dimensions of the sealing member 18 may be varied within the scope of the present teachings and will be significantly dictated by the size of the liner 14.

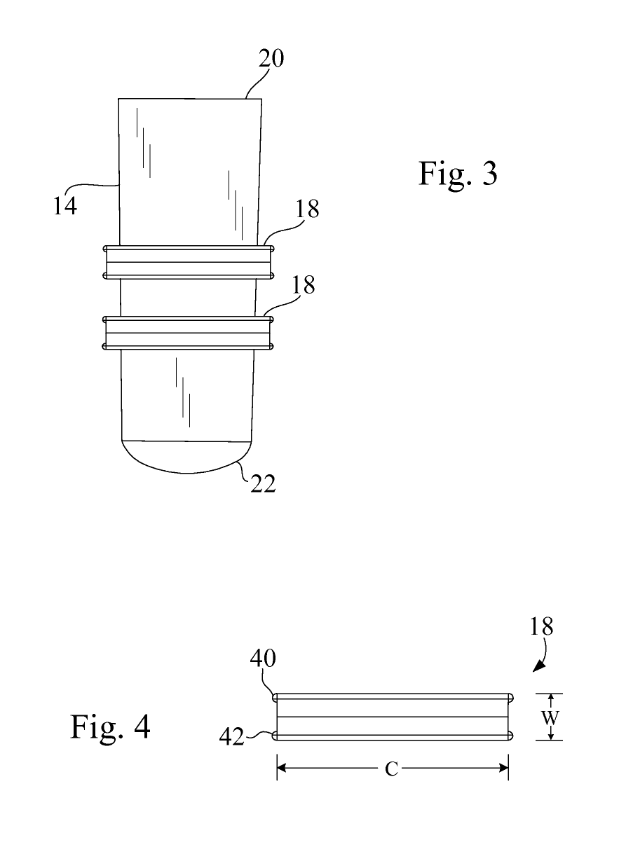

Turning to FIG. 3, the prosthesis 10 is shown to include a plurality of sealing members 18. In the particular embodiment illustrated, the prosthesis 10 includes two (2) sealing members 18. In other applications, the prosthesis 10 may include a greater number of sealing members 18. In certain applications, multiple sealing members 18 may be layered with respect to one another.

The one or more sealing member 18 may be secured to the liner 14 prior to attachment of the shell 16. Installation of each sealing member 18 may include the initial step of placing the liner 14 on the patient and temporarily taping the sealing member 18 to the liner 14 for fitting purposes. The sealing member 18 may have to be repositioned to determine the appropriate location for optimal comfort and function. Once the desired location is determined, the outer surface of the liner 14 is marked. The liner 14 may be removed and placed on a patient's cast model.

In a next step, the sealing member 18 is placed on the liner 14 and positioned to match up with markings. Colored tape may be wrapped around the liner 14 to closely follow proximal and distal borders of sealing member 18. Use of wider tape may help protect the liner surface from excess glue.

The sealing member 18 is next removed and a bead of adhesive is provided in the area between the two rings of tape. The adhesive is smoothed to evenly distribute it over the area between the two rings of tape. The sealing member 18 is placed over the area of glue and moved into position. The sealing member 18 may be gently pressed and smoothed into place and allowed to dry.

The tape borders may be removed and a thin bead of adhesive may be squeezed to fill the proximal area where sealing member 18 and liner surface meet. The bead of adhesive may be smoothed by finger to blend and create a finished edge. This step may be repeated on the distal border of sealing member 18.

The liner 14 may be secured to the patient in a generally conventionally manner. The hollow shell 16 is inserted over the liner 14. The shell 16 may slightly compress the sealing member 18 to provide a seal between the liner 14 and the shell 16. In certain applications, a cream (e.g., Nivea® or other) may be lightly applied to the sealing member 18 prior to attachment of the shell 16 (e.g., wet fit). As the shell 16 is pulled into place, a seal is created. Air trapped between the liner 14 and the shell 16 may be expelled through a valve in the shell 16.

While specific examples have been described in the specification and illustrated in the drawings, it will be understood by those skilled in the art that various changes may be made and equivalence may be substituted for elements thereof without departing from the scope of the present teachings as defined in the claims. Furthermore, the mixing and matching of features, elements and/or functions between various examples may be expressly contemplated herein so that one skilled in the art would appreciate from the present teachings that features, elements and/or functions of one example may be incorporated into another example as appropriate, unless described otherwise above. Moreover, many modifications may be made to adapt a particular situation or material to the present teachings without departing from the essential scope thereof. Therefore, it may be intended that the present teachings not be limited to the particular examples illustrated by the drawings and described in the specification as the best mode of presently contemplated for carrying out the present teachings but that the scope of the present disclosure will include any embodiments following within the foregoing description and any appended claims.

What is claimed is:

1. A limb prosthesis comprising:
a liner;
a shell; and
at least one sealing member secured to and circumferentially surrounding the liner, the at least one sealing member disposed between the liner and the shell;
wherein the at least one sealing member enhances security of the attachment of the shell to the liner.

2. The liner of a limb prosthesis of claim 1, wherein the at least one sealing member includes a plurality of sealing members.

3. The liner of a limb prosthesis of claim 1, wherein the at least one sealing member has a toroidal shape.

4. The liner of a limb prosthesis of claim 2, wherein the plurality of sealing members are oriented parallel to one another.

5. The liner of a limb prosthesis of claim 1, wherein the at least one sealing member is constructed of a flexible and stretchable material.

6. The line of a limb prosthesis of claim 1, wherein the at least one sealing member is constructed of silicone.

7. The liner of a limb prosthesis of claim 1, wherein each sealing member of the at least one sealing member includes at least one circumferentially existing rib.

8. A limb prosthesis comprising:
a liner;
a hollow shell having a proximal end and a distal end, the proximal end being open to receive the liner; and
at least one band-shaped sealing member attached to and circumferentially surrounding the liner, the at least one sealing member disposed between the liner and the shell.

9. The limb prosthesis of claim 8, wherein the at least one sealing member includes a plurality of sealing members.

10. The limb prosthesis of claim 8, wherein the at least one sealing member has a width significantly greater than a thickness.

11. The limb prosthesis of claim 9, wherein the plurality of sealing members are oriented generally parallel to one another.

12. The liner of a limb prosthesis of claim 8, wherein the at least one sealing member is constructed of a flexible and stretchable material.

13. The line of a limb prosthesis of claim 8, wherein the at least one sealing member is constructed of silicone.

14. The liner of a limb prosthesis of claim 8, wherein each sealing member of the at least one sealing member includes at least one circumferentially existing rib.

15. A method of securing a limb prosthesis to a patient, the method comprising:
securing a liner to a patient;
placing at least one band circumferentially around the liner; and
attaching a hollow shell to the liner by placing the hollow shell at least partially over the liner such that the at least one band is between the liner and the hollow shell and forms a seal therebetween.

16. The method of securing a limb prosthesis to a patient of claim 9, wherein placing at least one band circumferentially around the liner includes placing a plurality of bands circumferentially around the liner.

17. The liner of a limb prosthesis of claim 15, wherein the at least one sealing member is constructed of a flexible and stretchable material.

18. The line of a limb prosthesis of claim 15, wherein the at least one sealing member is constructed of silicone.

19. The liner of a limb prosthesis of claim 15, wherein each sealing member of the at least one sealing member includes at least one circumferentially existing rib.
